(19) United States
(12) Patent Application Publication
(10) Pub. No.: US 2007/0060989 A1
(43) Pub. Date: Mar. 15, 2007

(54) Apparatus and method for disrupting subcutaneous structures

(76) Inventors: Mark E. Deem, Mountain View, CA (US); Hanson Gifford, Woodside, CA (US)

Correspondence Address: FULWIDER PATTON - CABOCHON AESTHETICS, 6060 CENTER DRIVE, TENTH FLOOR, LOS ANGELES, CA, 90045, US

(21) Appl. No.: 11/515,634

(22) Filed: Sep. 5, 2006

Related U.S. Application Data
(60) Provisional application No. 60/715,398, filed on Sep. 7, 2005.

(51) Int. Cl. A61F 7/00; A61F 7/12

(52) U.S. Cl. 607/99

(57) ABSTRACT

Methods and apparatus are provided for disruption/destruction of subcutaneous structures in a mammalian body for the treatment of skin irregularities, and other disorders such as excess adipose tissue, cellulite, and scarring. Devices and methods include energy mediated applicators, microneedles, catheters and subcutaneous treatment devices for applying a treatment non-invasively through the skin, less invasively through the skin, or minimally invasively via a subcutaneous approach. Various agents to assist or enhance the procedures are also disclosed.

### CROSS-REFERENCES TO RELATED APPLICATIONS

This application claims priority to U.S. Provisional Patent Application Ser. No. 60/715,398 filed Sep. 7, 2005 the entirety of which is incorporated herein by reference.

### BACKGROUND OF THE INVENTION

The present invention relates to methods and apparatus for the treatment of dermal and subdermal skin irregularities, and more particularly, methods and apparatus are provided for disruption/destruction of subcutaneous structures in a mammalian body for the treatment of skin irregularities, and other disorders such as excess adipose tissue, cellulite, and scarring.

All publications and patents or patent applications mentioned in this specification are herein incorporated by reference to the same extent as if each individual publication, patent or patent application was specifically and individually so incorporated by reference.

Gynoid lipodystrophy is a localized metabolic disorder of the subcutaneous tissue which leads to an alteration in the topography of the cutaneous surface (skin), or a dimpling effect caused by increased fluid retention or proliferation of adipose tissue in certain subdermal regions. This condition, commonly known as cellulite, affects over 90% of most post-pubescent women, and some men. Cellulite commonly appears on the hips, buttocks and legs, but is not necessarily caused by being overweight, as is a common perception. Cellulite is formed in the subcutaneous level of tissue below the epidermis and dermis layers. In this region, fat cells are arranged in chambers surrounded by bands of connective tissue called septae. As water is retained, fat cells held within the perimeters defined by these fibrous septae expand and stretch the septae and surrounding connective tissue. Eventually this connective tissue contracts and hardens (scleroses) holding the skin at a non-flexible length, while the chambers between the septae continue to expand with weight gain, or water gain. This results in areas of the skin being held down while other sections bulge outward, resulting in the lumpy, ‘orange peel’ or ‘cottage-cheese’ appearance on the skin surface.

Even though obesity is not considered to be a root cause of cellulite, it can certainly worsen the dimpled appearance of a cellulitic region due to the increased number of fat cells in the region. Traditional fat extraction techniques such as liposuction that target deep fat and larger regions of the anatomy, can sometimes worsen the appearance of cellulite since the subdermal fat pockets remain and are accentuated by the loss of underlying bulk (deep fat) in the region. Many times liposuction is performed and patients still seek therapy for remaining skin irregularities, such as cellulite.

A variety of approaches for treatment of skin irregularities such as cellulite and removal of unwanted adipose tissue have been proposed. For example, methods and devices that provide mechanical massage to the affected area, through either a combination of suction and massage or suction, massage and application of energy, in addition to application of various topical agents are currently available. Developed in the 1950's, mesotherapy is the injection of various treatment solutions through the skin that has been widely used in Europe for conditions ranging from sports injuries to chronic pain, to cosmetic procedures to treat wrinkles and cellulite. The treatment consists of the injection or transfer of various agents through the skin to provide increased circulation and the potential for fat oxidation, such as aminophylline, hyaluronic acid, novocaine, plant extracts and other vitamins. The treatment entitled Aethyderm (Turnwood International, Ontario, Canada) employs a roller system that electroporates the stratum corneum to open small channels in the dermis, followed by the application of various mesotherapy agents, such as Vitamins, antifibrotics, lypolitics, anti-inflammatories and the like.

Massage techniques that improve lymphatic drainage were tried as early as the 1930's. Mechanical massage devices, or Pressotherapy, have also been developed such as the “Endermologie” device (LPG Systems, France) described further in U.S. Pat. Nos. 5,885,232 and 5,961,475, hereby incorporated by reference in their entirety, the “Synergie” device (Dynatronics, Salt Lake City, Utah) and the “Silklight” device (Lumenis, Tel Aviv, Israel) described in United States Patent Publication US2005/0049543, incorporated by reference in its entirety, all utilizing subdermal massage via vacuum and mechanical rollers. Other approaches have included a variety of energy sources, such as Cynosure's “TriActive” device (Cynosure, Westford, Mass.) utilizing a pulsed semiconductor laser in addition to mechanical massage, and the “Cellulux” device (Palomar Medical, Burlington, Mass.) which emits infrared light through a cooled chiller to target subcutaneous adipose tissue. The “VelaSmooth” system (Syneron, Inc., Yokneam Illit, Israel) detailed in U.S. Pat. Nos. 6,889,090, 6,702,808 and 6,662,054, incorporated by reference in their entirety, employs bipolar radiofrequency energy in conjunction with suction to increase metabolism in adipose tissue, and the “Thermacool” device (Thermage, Inc., Hayward, Calif.) utilizes radiofrequency energy to shrink the subdermal fibrous septae to treat wrinkles and other skin defects, as detailed in U.S. Pat. Nos. 5,755,753, 6,749,624, 5,948,011, 6,387,380, 6,381,497, 6,381,498,5,919,219, 3,377,854, 6,377,855, 6,241,753, 6,405,090, 6,311,090 5,871,524, 6,413,255, 6,461,378, 6,453,202, 6,430,446, incorporated herein by reference in their entirety. Other energy based therapies such as electrolipophoresis, using several pairs of needles to apply a low frequency interstitial electromagnetic field to aid circulatory drainage have also been developed (“Cellulite. Aspects of Cliniques et Morpho-histologiques”, J. med. Esth. Et Chir Derm (1983); 10(40), 229-223), hereby incorporated by reference in its entirety. Similarly, non-invasive ultrasound is used in the “Dermosonic” device (Symedex Medical, Minneapolis, Minn.) to promote reabsorption and drainage of retained fluids and toxins. Further, United States Patent Application US2004/0019371 depicts the application of energy to modify cells to treat skin irregularities, and United States Patent Application US2003/0220674 describes the use of cooling to treat cellulite.

Another approach to the treatment of skin irregularities such as scarring and dimpling is a technique called subcision. This technique involves the insertion of a relatively large gauge needle subdermally in the region of dimpling or scarring, and then mechanically manipulating the needle below the skin to break up the fibrous septae in the subdermal region. As detailed in “Subcision: A treatment for cellulite”, International Journal of Dermatology (2000) 39:539-544, a local anesthetic is injected into the targeted region, and an 18 gauge needle is inserted 10-20 mm below the cutaneous surface. The needle is then directed parallel to the epidermis to create a dissection plane beneath the skin to essentially tear through, or “free up” the tightened septae causing the dimpling or scarring. Pressure is then applied to control bleeding acutely, and then by the use of compressive clothing following the procedure. While clinically effective in some patients, pain, bruising, bleeding and scarring can result. U.S. Pat. No. 6,916,328, incorporated by reference in its entirety, describes a laterally deployed cutting mechanism for subcision, and a technique employing an ultrasonically assisted subcision technique is detailed in “Surgical Treatment of Cellulite and its Results”, American Journal of Cosmetic Surgery, (1999)16:4 299-303, the contents of which are incorporated herein by reference.

Certain other techniques known as liposuction, tumescent liposuction, lypolosis and the like, target adipose tissue in the subdermal and deep fat regions of the body. These techniques may include also removing the fat cells once they are disrupted, or leaving them to be resorbed by the body's immune/lymphatic system. Traditional liposuction includes the use of a surgical cannula placed at the site of the fat to be removed, and then the use of infusion of fluids and mechanical motion of the cannula to break up the fatty tissue, and suction to “vacuum” the disrupted fatty tissue directly out of the patient. The “Lysonix” system (Mentor Corporation, Santa Barbara, Calif.) utilizes an ultrasonic transducer on the handpiece of the suction cannula to assist in tissue disruption (by cavitation of the tissue at the targeted site), as further detailed in U.S. Pat. Nos. 4,886,491 and 5,419,761, incorporated herein by reference in their entirety. In addition, cryogenic cooling has been proposed for destroying adipose tissue as detailed in U.S. Pat. Nos. 6,041,787 and 6,032,675, incorporated herein in their entirety. A variation on the traditional liposuction technique known as tumescent liposuction was introduced in 1985 and is currently standard of care in the United States. It involves the infusion of tumescent fluids to the targeted region prior to mechanical disruption and removal by the suction cannula. The fluids help to ease the pain of the mechanical disruption, while also swelling the tissues making them more susceptible to mechanical removal. Various combinations of fluids may be employed in the tumescent solution including a local anesthetic such as lidocaine, a vasoconstrictive agent such as epinephrine, saline, potassium and the like. The benefits of such an approach are detailed in the following articles, “Laboratory and Histopathologic Comparative Study of Internal Ultrasound-Assisted Lipoplasty and Tumescent Lipoplasty” Plastic and Reconstructive Surgery, September 15, (2002) 110:4, 1158-1164, and “When One Liter Does Not Equal 1000 Milliliters: Implications for the Tumescent Technique” Dermatol. Surg. (2000) 26:1024-1028, the contents of which are expressly incorporated herein by reference in their entirety.

Liposonix (Bothell, Wash.) and Ultrashape (TelAviv, Israel) employ the use of focused ultrasound to destroy adipose tissue noninvasively. U.S. Pat. No. 6,607,498 and United States Patent Publications US2004/0106867 and US2005/0154431, incorporated by reference in their entirety, depict these systems.

Various other approaches employing dermatologic creams, lotions, vitamins and herbal supplements have also been proposed. Private spas and salons offer cellulite massage treatments that include body scrubs, pressure point massage, essential oils, and herbal products using extracts from plant species such as seaweed, horsetail and clematis and ivy have also been proposed. Although a multitude of therapies exist, most of them do not provide a lasting effect on the skin irregularity, and for some, one therapy may cause the worsening of another (as in the case of liposuction causing scarring or a more pronounced appearance of cellulite), or have negative side effects that limit its adoption. Most therapies require multiple treatments on an ongoing basis to maintain their effect at significant expense and with mixed results.

In light of the foregoing, it would be desirable to provide methods and apparatus for treating skin irregularities and to provide a sustained aesthetic result to a body region, such as the face, neck, arms, legs, thighs, buttocks, breasts, stomach and other targeted regions which are minimally or non-invasive.

It would also be desirable to provide methods and apparatus for treating skin irregularities that enhance prior techniques and make them less invasive and subject to fewer side effects.

### SUMMARY OF THE INVENTION

In view of the foregoing, one aspect of the present invention is to provide methods and apparatus for treatment of dermal and subdermal skin irregularities, and more particularly, treatment of excess adipose tissue, cellulite, scarring and related disorders which are minimally or non-invasive, controlled and selective, and offer a more durable effect.

In one aspect of the present invention methods and apparatus are provided for treating such conditions by applying devices non-invasively (on the skin surface), less invasively (between 3 and 10 mm below the dermal surface), or minimally invasively (6 mm and deeper to the deeper fat layers) to provide disruption/destruction of subcutaneous structures in a mammalian body by utilizing an electric, ultrasonic or other energy field.

In a further aspect of the invention, such energy fields may be generated by a pulse or pulses of a designated duration and amplitude to disrupt tissue at the cellular level via permeabilization of the targeted cell membrane. In a further aspect of the invention, it may be desirable to cause irreversible cell damage by the creation of pores in the cell membrane of the targeted subcutaneous structure which result in the death of the cell.

In another aspect of the present invention it may be desirable to disrupt subcutaneous structures utilizing the devices and methods of the present invention through the application of radiofrequency energy, direct current, resistive heat energy, ultrasound energy, microwave energy or laser energy.

In another aspect of the invention, electromanipulation of the targeted tissue (such as connective tissue, collagen, adipose tissue or the like) may be enhanced by the injection or application of an enhancing agent, such as hypotonic saline, potassium and the like to change the intracellular environment and/or cellular membrane so as to make it more susceptible to the applied electric field to disrupts the tissue at the cellular level via causing reversible or irreversible electroporation of the cellular membrane.

In another aspect of the invention, disruption of targeted tissue (such as connective tissue, collagen, adipose tissue or the like) may be enhanced by the injection or application of an enhancing agent, such as microbubbles, agitated saline, commercially available ultrasound contrast agent or the like to increase the energy delivered to the area and enhance the therapeutic effect, such as by cavitation.

In a further aspect of the present invention, energy transmission members may be placed dermally, transdermally or subdermally, as appropriate, to enhance the delivery of energy to the targeted site.

A further aspect of the invention is to provide methods and apparatus for treating skin irregularities and other related disorders by utilizing any of the energy approaches of the present invention in conjunction with application of a treatment enhancing agent to the treatment site, such as a lidocaine, epinephrine, hypotonic saline, potassium, agitated saline, microbubbles, commercially available ultrasound contrast agents, microspheres, or the like.

In addition, once the treatment of the present invention has been applied, it is another aspect of the invention to apply filling agents such as adipocytes, fat, PLLA, collagen, hydroxyapetite, hyalluonic acid, or the like as needed to enhance the overall desired effect.

In a further aspect of the invention, it may be desirable to provide methods and devices that selectively disrupt certain cell types and not others, to provide a therapy that can be applied safely from multiple locations within the body.

One aspect of the present invention is a medical device for disrupting subcutaneous tissue, including an electrical field generator, at least two electrodes electrically connected with the electrical field generator, and an injection module configured to inject a treatment enhancing solution into the subcutaneous tissue to be treated. The at least one electrode is adapted for insertion into the subcutaneous tissue to be treated and at least one other electrode is adapted for application to the epidermis of a patient to be treated. In yet another aspect of the invention, at least two electrodes are adapted for application to the epidermis of a patient to be treated. The at least two electrodes may be adapted for insertion into the subcutaneous tissue to be treated. One of the at least two electrodes may be configured as a ground electrode. The at least two electrodes may be configured as bipolar electrodes. At least one of the at least two electrodes may be generally torroidal in shape. At least one of the at least two electrodes may be generally cylindrically shaped. In still another aspect, the electrical field generator is an electroporation generator.

The medical device may further include a housing, wherein one of the at least two electrodes is disposed in the housing. At least one electrode may be configured as a central treatment element disposed in the housing, and an annular area may be disposed between the central treatment element and the housing. The annular region may be configured for connection with a source of negative pressure, whereby the housing is adapted for contact with the skin overlying the area to be treated. The central treatment element may be recessed into the housing. The central treatment element may further be adapted to roll over the skin of a patient to be treated.

In a further aspect of the present invention, the device includes a pad having microneedles connected to the injection module, wherein the pad is adapted to conform to the skin of a patient to be treated. The pad may include a reservoir and an actuation element for deploying the microneedles. In still a further aspect of the invention, at least one of the microneedles is configured as one of the at least two electrodes.

In yet a further aspect of the invention a catheter device may be adapted to deploy tines to a subcutaneous region to be treated. The tines are selected from the group consisting of needles, electrodes, and cutting elements.

Yet another aspect of the invention is a subcutaneous tissue disruption device, including a tubular element having a first proximal end, a second distal end adapted for insertion into subcutaneous tissue, and a channel longitudinally disposed therebetween. A plurality of extendable elongated elements having first proximal ends and second distal ends are disposed within the channel and capable of movement from a first retracted configuration within the channel to a second extended configuration outside of the channel, wherein the distal ends of the elongated elements are farther apart from each other in the extended configuration than in the retracted configuration. The plurality of extendable elongated elements may be selected from the group consisting of needles, electrodes, and cutting elements. In yet a further aspect of the invention, the plurality of extendable elongated elements are geometrically configured to shape an energy field for a biological tissue disruption effect.

One aspect of the present invention is a method for selective disruption of subcutaneous structures, including providing a first electrode and a second electrode, placing the first electrode adjacent to the tissue to be treated, connecting the first electrode and the second electrode to an energy delivery system, the energy delivery system being configured to produce an electrical current between the first and the second electrode, and providing electrical current between the first electrode and the second electrode, thereby increasing permeability of at least one cell. At least the first electrode may be geometrically configured to shape an energy field for a biological tissue disruption effect. The method may further include rolling a central treatment element disposed within a housing over the tissue to be treated, wherein the first electrode is disposed in the central treatment element. In still another aspect of the invention, less than atmospheric pressure is provided to an annular area disposed around the central treatment element.

### BRIEF DESCRIPTION OF THE DRAWINGS

Further features of the invention, its nature and various advantages will be more apparent from the accompanying drawings and the following detailed description, in which:

FIG. 1A is an illustration of various layers of a normal region of cutaneous and subcutaneous tissues;

FIG. 1B is an illustration of various layers of an abnormal region of cutaneous and subcutaneous tissues;

FIG. 2 is an illustration of one embodiment of a device of the present invention for non-invasive energy application;

FIG. 3A is a schematic illustration of a clamp embodiment of the present invention;

FIG. 3B is a schematic illustration of a model showing current distribution of the clamp embodiment of FIG. 3A;

FIG. 4 is a schematic illustration of the clamp embodiment of FIG. 3A using a cylindrical electrode;

FIG. 5A is a schematic illustration of a roller ball embodiment of the present invention;

FIG. 5B is a schematic illustration showing current distribution of the roller ball embodiment of FIG. 5A;

FIG. 6 illustrates an embodiment of an injection system of the present invention, including an externally applied energy applicator used in conjunction with a treatment enhancing agent;

FIG. 7A illustrates an embodiment of the present invention having a system including a pad having microneedles for application to a skin surface;

FIG. 7B illustrates the embodiment of FIG. 7A wherein suction has pulled the epidermal surface up towards the pad resulting in the microneedles penetrating the skin surface;

FIG. 8A is a cross sectional view of the embodiment of FIG. 7A applied to targeted tissues;

FIG. 8B illustrates one embodiment of a device of the present invention having microneedles disposed on a pad in an array;

FIG. 8C is an enlarged view of a portion of the device of FIG. 8B;

FIG. 9 depicts an embodiment of an interstitial electrode array of the present invention;

FIG. 10 depicts an embodiment of an interstitial electrode array of the present invention;

FIG. 11 depicts an embodiment of an interstitial electrode array of the present invention;

FIG. 12 illustrates an embodiment of an interstitial electrode array of the present invention used in conjunction with an electrode applied to a skin surface;

FIG. 13 illustrates an embodiment of an interstitial electrode array of the present invention used in conjunction with an electrode applied to a skin surface;

FIG. 14 illustrates an interstitial electrode of the present invention used in conjunction with an electrode applied to a skin surface and a proposed treatment layout;

FIG. 14A illustrates an example of an electrode treatment layout;

FIG. 15 illustrates a template for use with individually placed energy transmission elements;

FIG. 16 illustrates an example of a treatment algorithm for use with multiple energy transmission elements;

FIG. 17A illustrates a system and device for applying energy while injecting a treatment enhancement agent;

FIG. 17B illustrates the system and device of FIG. 17A wherein the handpiece is inserted into the tissue to be treated; and

FIG. 18 illustrates an assembly for treating subcutaneous tissues.

### DETAILED DESCRIPTION OF THE PREFERRED EMBODIMENTS

The present invention is related to methods and apparatus for targeting and disrupting subcutaneous structures, such as collagen, connective tissue, adipose tissue (fat cells) and the like (collectively “target tissue” or “subcutaneous structures”). The present invention is useful for improving the aesthetic appearance of the targeted region. Targeted regions may consist of any surface or contour of the human form that it is desirable to enhance, including the face, chin, neck, chest, breasts, arms, torso, abdominal region (including pelvic region), thighs, buttocks, knees and legs. The target tissue may include the connective tissue or septae of the region, or the underlying tissues that may exacerbate the unwanted body contour, such as subdermal and deeper fat deposits or layers.

Skin irregularities refer to conditions that decrease a person's satisfaction with their outward appearance, such as cellulite, scarring, or fat deposits or excess fat in certain regions, such as neck, chin, breasts, hips, abdomen, arms and the like.

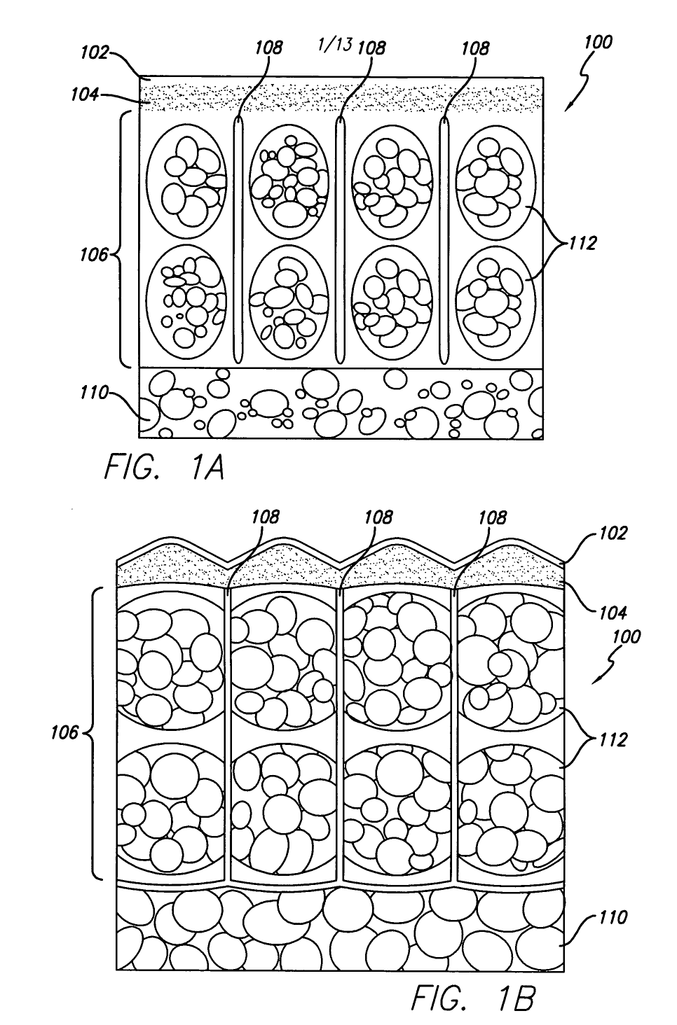

Referring now to FIGS. 1A and 1B, a cross section of the targeted region 100 of cutaneous tissues and/or subcutaneous tissues to be treated is shown, including the epidermis 102, dermis 104, subcutaneous fat 106, fibrous septae 108, microcirculation, lymph drainage, and deeper fat layers 110. The dermis interfaces with the fatty subcutaneous connective tissue that attaches to the dermal layers via substantially vertical septae or collagenous fibers. The subcutaneous fatty tissue is compartmentalized into chambers 112 of adipose tissue or fat, separated by the fibers of the septae. These chambers can swell due to the presence of increased adipocytes or retained fluid which causes tension on the septae and ultimately dimpling at the skin surface as the fatty regions swell and the septae thicken under the tension. Microcirculation and lymphatic drainage may then become impaired, further exacerbating the local metabolic pathology. FIG. 1A illustrates a fairly normal skin cross section, not exhibiting skin irregularities. FIG. 1B illustrates a subcutaneous fat layer that is swollen and septae tightened, leading the to an irregular skin surface characteristic.

A reserve or deeper fat layer 110 is disposed beneath the subcutaneous fat layer 106 and may also contribute to a skin irregularity, so for those purposes, it is considered a “subcutaneous structure” for purposes of this disclosure. In at least one embodiment, devices of the present invention may be directed to targeted regions 100 such as those described above. Some particular examples include, energy assisted subcision, disruption of the fibrous septae 108, disruption of the subcutaneous fat 106 cells to lessen the outward pressure on the skin surface that contributes to dimpling, or disruption of a deeper fat layer 110 for overall surface contouring.

To achieve the goals of the present invention, it may be desirable to employ methods and apparatus for achieving disruption of subcutaneous structures 106, 108, 110 utilizing a variety of energy modalities, including electroporation (reversible and/or irreversible), pulsed electric fields, radiofrequency energy, microwave energy, laser energy, ultrasonic energy and the like. For example, the application of pulsed electric fields and/or electroporation applied directly to the targeted region 100 or in proximity to the targeted region can produce the desired disruption. For purposes of this disclosure, the term “electroporation” can encompass the use of pulsed electric fields (PEFs), nanosecond pulsed electric fields (nsPEFs), ionophoreseis, electrophoresis, electropermeabilization, sonoporation and/or combinations thereof, permanent or temporary, reversible or irreversible, with or without the use of adjunctive agents, without necessitating the presence of a thermal effect. Similarly, the term “electrode” used herein, encompasses the use of various types of energy producing devices, including antennas, for example, microwave transmitters, and ultrasonic elements.

Reversible electroporation, first observed in the early 1970's, has been used extensively in medicine and biology to transfer chemicals, drugs, genes and other molecules into targeted cells for a variety of purposes such as electrochemotherapy, gene transfer, transdermal drug delivery, vaccines, and the like. Irreversible electroporation, although avoided for the most part historically when using electroporation techniques, has more recently been proposed for cell separation in such applications as debacterilization of water and food, stem cell enrichment and cancer cell purging (U.S. Pat. No. 6,043,066 to Mangano), directed ablation of neoplastic prostate tissues (US2003/0060856 to Chornenky), treatment of restenosis in body vessels (US2001/0044596 to Jaafar), selective irreversible electroporation of fat cells (US 2004/0019371 to Jaafar) and ablation of tumors (Davalos, et al Tissue Ablation with Irreversible Electroporation, Annals of Biomedical Engineering 33:2, pp. 223-231 (February 2005), the entire contents of each are expressly incorporated herein by reference.

Further, energy fields applied in ultrashort pulses, or nanosecond pulsed electric fields (nsPEFs) have application to the present invention. Such technology utilizes ultrashort pulse lengths to target subcellular structures without permanently disrupting the outer membrane. An example of this technology is described by Schoenbach et al. in Intracellular Effect of Ultrashort Electrical Pulses in J. Bioelectromagnetics 22:440-448 (2001), and further described in U.S. Pat. No. 6,326,177, the contents of which is expressly herein incorporated by reference. The short pulses target the intracellular apparatus, and although the cell membrane may exhibit an electroporative effect, such effect may be reversible and may not lead to permanent membrane disruption. Following application of nanosecond pulses apoptosis is induced in the intracellular contents, affecting the cell's viability (for example the ability to reproduce).

In general, electroporation may be achieved utilizing a device adapted to activate an electrode set or series of electrodes to produce an electric field. Such a field can be generated in a multipolar, bipolar, or monopolar electrode configuration. When applied to cells, depending on the duration and strength of the applied pulses, this field operates to increase the permeabolization of the cell membrane and either 1) reversibly open the cell membrane for a short period of time by causing pores to form in the cell lipid bilayer allowing entry of various therapeutic elements or molecules, after which, when energy application ceases, the pores spontaneously close without killing the cell, or 2) irreversibly opening or porating the cell membrane causing cell instability resulting in cell death utilizing higher intensity (longer or higher energy) pulses, or 3) applying energy in nanosecond pulses resulting in disruption of the intracellular matrix leading to apoptosis and cell death, without causing irreversible poration of the cellular membrane. As characterized by Weaver, Electroporation: A General Phenomenon for Manipulating Cells and Tissues Journal of Cellular Biochemistry, 51:426-435 (1993), the entirety of which is incorporated herein by reference, short (1-1100 s) and longer (1-10 ms) pulses have induced electroporation in a variety of cell types. In a single cell model, most cells will exhibit electroporation in the range of 1-1.5V applied across the cell (membrane potential). For applications of electroporation to cell volumes, ranges of 10 V/cm to 10,000 V/cm and pulse durations ranging from 1.0 nanosecond to 0.1 seconds can be applied

Certain factors affect how a delivered electric field will affect a targeted cell, including cell size, cell shape, cell orientation with respect to the applied electric field, cell temperature, distance between cells (cell-cell separation), cell type, tissue heterogeneity, properties of the cellular membrane and the like. Larger cells may be more vulnerable to injury. For example, skeletal muscle cells have been shown to be more susceptible to electrical injury than nearby connective tissue cells (Gaylor et al. Tissue Injury in Electrical Trauma, J. Theor. Biol. (1988) 133, 223-237), the entirety of which is incorporated herein by reference. Adipose tissue, or fat cells, may be less vulnerable to injury due to their insulative properties, and as such, may require pre-treatment or treatment during the application of energy to make the cell membrane more susceptible to damage.

According to research in the area, hypotonic solution can significantly increase human adipocyte cell diameter. Within fifteen minutes of injection, the effect of quarter normal saline has been reported as having a significant effect on cell diameter. Scientific Basis for Use of Hypotonic Solutions with Ultrasonic Liposuction (Jennifer M. Bennett, MS, abstract presented at Plastic Surgery 2004). For example, if fat cells are the target tissue in the present invention, it may be necessary to infuse a solution such as a hypotonic saline to the region which in turn produces adipocyte swelling that results in an increase in the stress on the cell membrane, making it more susceptible to disruption by electroporation, application of ultrasound energy, or application of other energy modalities. Such enhancing effects may be a change in cell size, increased cellular conductivity, increased extracellular conductivity, increased wall stress, leading to increased permeability. In addition, modifying the concentrations of saline, potassium and other ingredients in the solution may affect cell membrane permeabolization.

In addition, how cells are oriented within the applied field can make them more susceptible to injury, for example, when the major axis of nonspherical cells is oriented along the electric field, it is more susceptible to rupture (Lee et al, Electrical Injury Mechanisms: Electrical Breakdown of Cell Membranes, Plastic and Reconstructive Surgery, November 1987, 672-679, the entirety of which is incorporated herein by reference.) For example, in the context of the present invention, depending on the orientation of the connective tissue it may be more or less susceptible to a given energy field depending on the direction of the field. Various waveforms or shapes of pulses may be applied to achieve electroporation, including sinusoidal AC pulses, DC pulses, square wave pulses, exponentially decaying waveforms or other pulse shapes such as combined AC/DC pulses, or DC shifted RF signals such as those described by Chang in Cell Poration and Cell Fusion using an Oscillating Electric Field, Biophysical Journal October 1989, Volume 56 pgs 641-652, the entirety of which is incorporated herein by reference, depending on the pulse generator used or the effect desired. The parameters of applied energy may be varied, including all or some of the following: waveform shape, amplitude, pulse duration, interval between pulses, number of pulses, combination of waveforms and the like. Electroporation catheter systems of the present invention may comprise a pulse generator such as those generators available from Cytopulse Sciences, Inc. (Columbia, Md.) or the Gene Pulser Xcell (Bio-Rad, Inc.), IGEA (Carpi, Italy), or Inovio (San Diego, Calif.), electrically connected to a energy application device such as a surface electrode or catheter electrode. The generator may be modified to produce a higher voltage, increased pulse capacity or other modifications to induce irreversible electroportion. In one embodiment, the generator may be current limited such that an e-field is allowed to stay longer, whereby cell electroporation in fat tissue may be enhanced and/or disruption of muscle tissue minimized.

According to one embodiment of the present invention, a variety of treatment enhancing agents 54 may be used in conjunction with the application of the various energy modalities, depending on the desired effects, some of which are detailed below. For example, agents may be transmitted transdermally, or via subcutaneous injection, either directly from the treatment device, or from a remote injection site, including intravenous delivery. Treatment enhancing agents may include, anesthetics such as lidocaine, vasoconstrictive agents such as epinephrine, hypotonic saline, potassium, agitated saline, microbubbles, commercially available ultrasound contrast agents, microspheres, adipocytes, fat, autologous tissues (e.g. lysed fat cells to produce clean adipocytes to form a tissue graft to minimize hostile response from the body), PLLA, and hydroxyappetite. Treatment enhancing agents may be delivered prior to, during or following the energy application treatment of the present invention.

Devices of the present invention include those that are applied non-invasively (on the skin surface or epidermis 102), less invasively (through the skin between about 3 and 10 mm below the epidermal surface 102), or minimally invasively (about 8 mm and deeper to deeper subcutaneous regions 106, and deeper fat layers 110) to provide disruption/destruction of subcutaneous structures 106, 108, 110 in a mammalian body by utilizing an energy field. Depending on the desired effect, the energy chosen and the electrode design can have impact on the type of structure that is successfully targeted. For purposes of this disclosure, certain energy modalities and electrode combinations are given, but are not intended to be limiting to the scope of the invention.

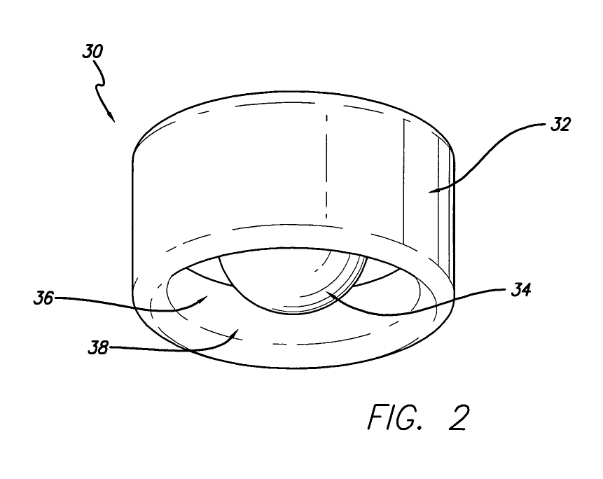

Referring now to FIG. 2, one embodiment of the present invention includes a device 30 having a housing 32 and a central treatment element 34. Disposed between the housing and the central treatment element is an annular region 36. The annular region includes an opening 38 that may measure between about 5 and 20 mm from the housing to the central treatment element. In one embodiment, the central treatment element is configured as a roller that may be rotatably connected within the housing to allow it to roll as the housing is moved over the skin surface 102 (FIG. 1). In yet another embodiment, the central treatment element may also be partially recessed into the housing, for example about 5-30 mm, but extends a sufficient distance such that when it is applied to the skin surface it compresses the skin to provide better contact for the electrical connection. In one embodiment, the central treatment element can be an electrode or the housing can be the electrode, with a ground (not shown) located somewhere on the patient's body in the form of a grounding pad (not shown), or the housing and the central treatment element can both be active electrodes to form a bipolar system. In one embodiment, both the housing and the central treatment element would contact the skin or epidermis 102 generally simultaneously while power is delivered. As described in more detail below, it may be advantageous to connect the device to a suction lumen by inserting a lumen in connection with the annular region to allow for suction when the device is applied to the skin. For example, in at least one embodiment, medical suction or negative pressure may be connected with the annular region 36 to pull the targeted region 100 (FIG. 1) and the device 30 against each other. This way, contact with the skin or epidermis 102 is maintained, and the desired compression achieved. The compression of the various tissue layers by the treatment device can impact the amount of energy required to achieve a therapeutic effect.

The central treatment element 34 or housing 32 may be configured in any number of shapes. In at least one embodiment, the central treatment element 34 may be shaped as a cylinder, a toroid, an ellipse or the like. In certain applications, a geometry with at least one radius of curvature is desirable to minimize the “edge effect” when energy is delivered and concentrated in one area of the treatment element.

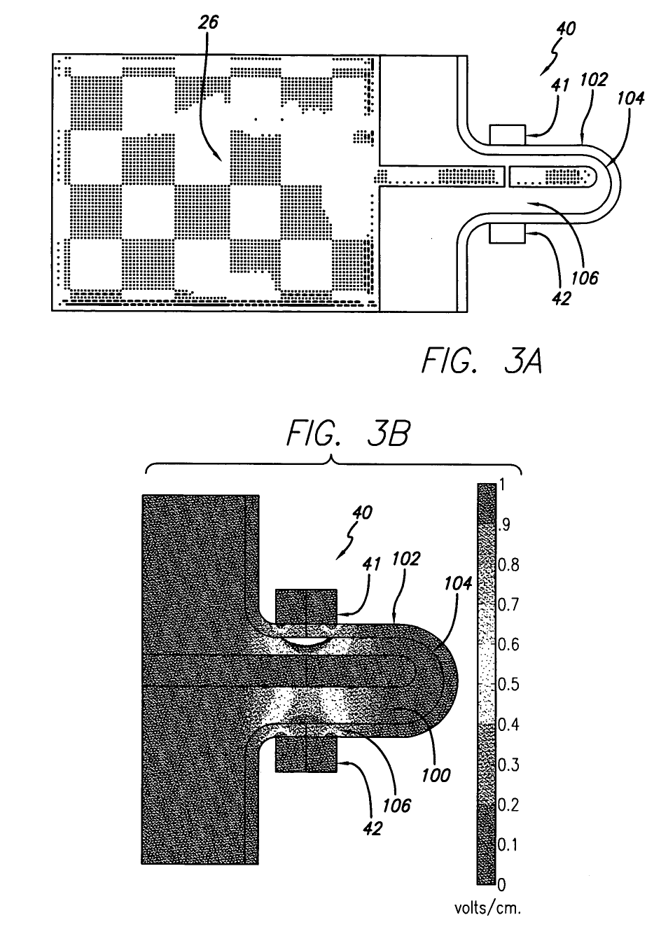

Referring now to FIG. 3A, another embodiment of the present invention includes a clamp 40 that is positioned on either side of a region of targeted tissue 100. In one embodiment, the clamp is configured in a bipolar configuration having a first electrode 41 and a second electrode 42 which contact the epidermis 102. The subcutaneous tissue 106 to be treated is disposed between the electrodes. The first electrode supplies a voltage and the second electrode is a ground.

Referring also now to FIG. 3B, the “edge effect” or concentration of current at the sharp edges of the clamp arm or electrode 40 show an increased energy density that is likely to be undesirable to the desired effect of the present invention where a more uniform energy delivery is most beneficial. A more uniform delivery of energy reduces the likelihood of premature impedance rise, that can reduce the amount and duration of energy delivered, or unintended tissue damage to surrounding structures, such as the epidermis 102 (FIG. 1).

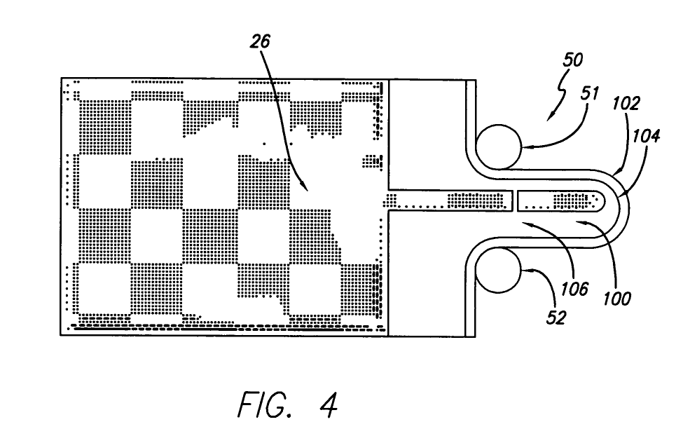

FIG. 4 depicts the cross section of another embodiment of the present invention, wherein a clamp 50 having generally cylindrical elements 51, 52 are employed to lessen the “edge effect” and make application of current more uniform. In this embodiment one arm 51 of the clamp is shown as the active electrode and the other arm 52 of the clamp as the ground, but in fact both clamp arms 51, 52 could be active electrodes and the ground (not shown) located on the tissue of the patient near the treated region 100, or remote from the treated region.

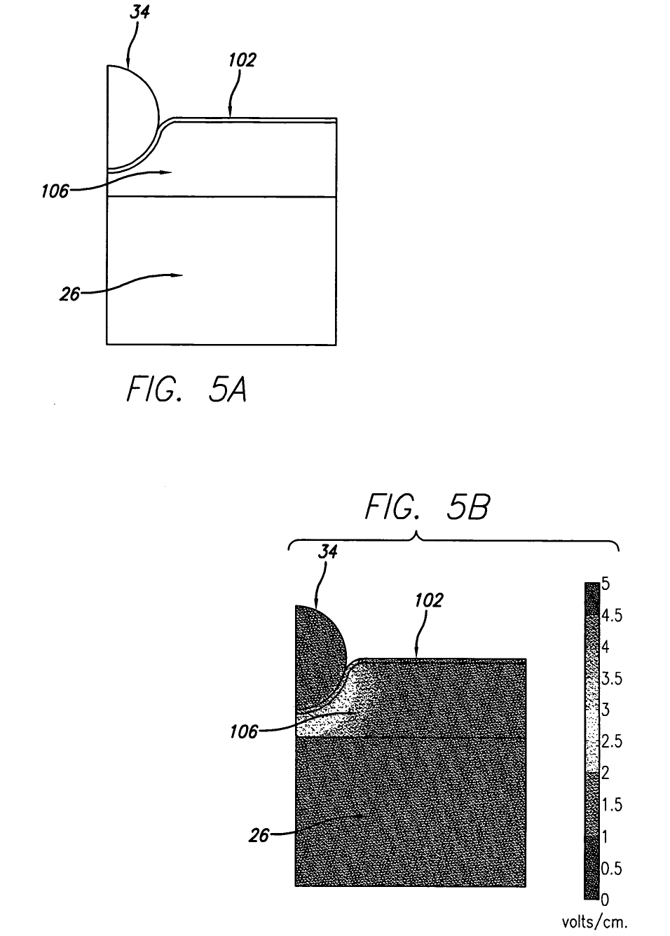

FIG. 5A illustrates application of the device 30 of FIG. 2 in a monopolar configuration, utilizing a central treatment element 34 toroidal in shape and measuring, for example, 45 mm in spherical diameter. In this embodiment, the central treatment element is the active electrode, positively charged, and the desired target tissue is subdermal fat 106 and connective tissue including the fibrous septae 108 and deep fat 110 (FIG. 1) up to the muscular layer. Various spherical diameters of central treatment elements can be used, for example 10 mm to 50 mm, or multiple small elements may be employed. Referring also now to FIG. 5B, there is a lack of “edge effect” present given the radius of curvature of the central treatment element, in addition to the targeted energy within the subdermal layers 106, 108, 110 (FIG. 1).

For exemplary purposes, the devices depicted in FIGS. 2A-5B may be employed using a variety of energy sources, but in particular with an electroporation generator, such as those earlier described. A variety of power may be delivered ranging from 5-2000 volts, and depending on thickness of the tissue and type of cell targeted, a field strength in the range of 50 to 10,000 V/cm, for example in the range of 100 to 3,000 V/cm. Such energy delivery may also be pulsed or switched to minimize muscle contraction while maximizing the disruptive effect to the target tissue.

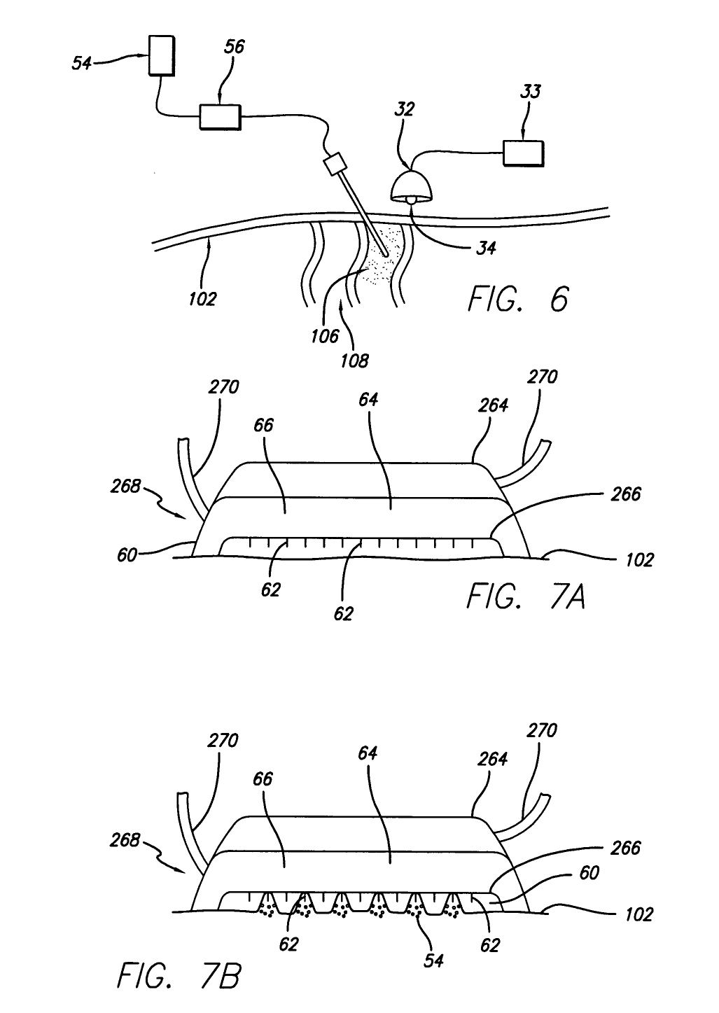

The energy application devices of the present invention may be used in conjunction with injectable enhancing agents 54, described in more detail elsewhere herein. Referring also now to FIG. 6, at least one embodiment of the invention includes a non-invasive energy delivery system having a central treatment element 34 used in conjunction with a subcutaneous injection of a treatment enhancing agent 54. In this embodiment, the energy delivery system 33 may be an electroporation generator as discussed above, and the central treatment element 34 an electrode, or it may be an ultrasound generator operatively connected to an ultrasound transducer such as those systems made by Siemens/Acuson (Malvern, Pa.). The injection may be targeted at any of the subcutaneous structures to be disrupted, including the subcutaneous fat 106, deep fat 110 (FIG. 1), fibrous septae 108 or other connective tissue to be disrupted. This system may also include an injector 56 that “foams” or agitates the solution prior to injection to produce increased energy potential at the treatment site, in the form of bubbles, etc. that explode when contacted with the energy applied from the skin surface.

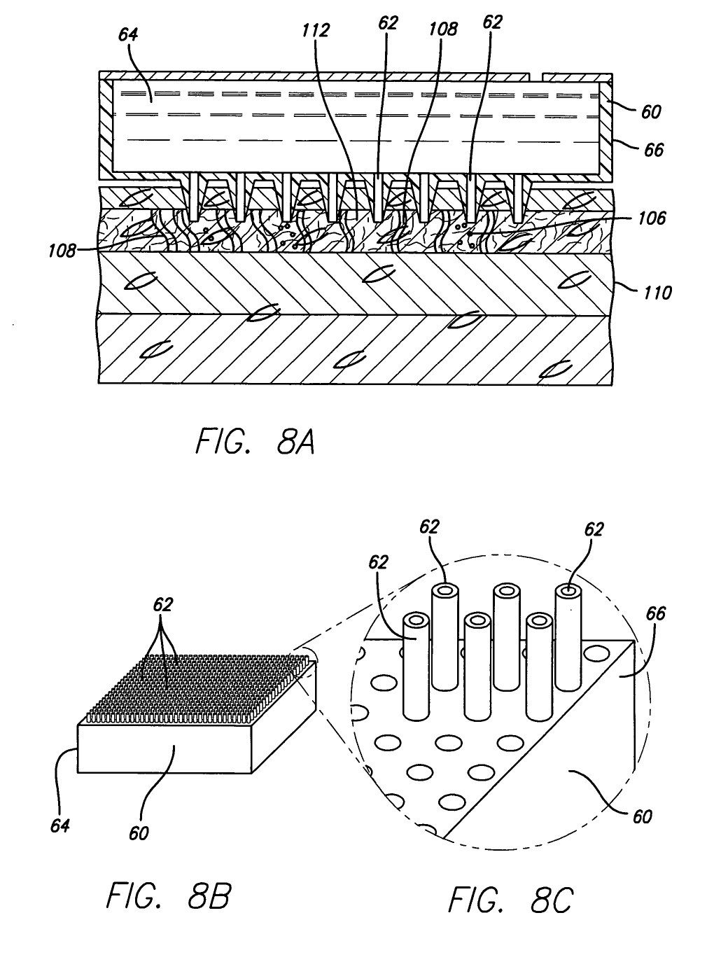

Referring now to FIGS. 7A-B and FIGS. 8A-C, in a further embodiment of the present invention a handpiece 268 includes a pad 60 that is capable of conforming to the skin surface of a patient. The pad contains a plurality of microneedles 62 extending therefrom, and is in communication with a reservoir 64. The device further comprises an actuation element 66, such as a bladder that can be distended with air or fluid to deploy the microneedles through the dermal layers of the patient's skin. Needle insertion through the skin can substantially reduce the resistance in the target tissue, making the targeted tissue more susceptible to the applied energy. The microneedles may extend into the skin a distance from 0.5 mm to 20 mm, depending on the target tissue to be treated, but in any event through stratum corneum. For example, to treat cellulite a depth of penetration from 1-5 mm may be desired, and for deeper subcutaneous fat, a depth of 3-20 mm, for example 5-10 mm. In one embodiment, all but the active portion of the microneedle shall be insulated to protect the skin from unwanted tissue damage, for example the first 0.5 mm to 1 mm may carry insulation. In yet another embodiment, the needles may be fully insulated. In still another embodiment, the needles are not insulated. The microneedles may be operatively connected to an energy source 33 (FIG. 6), such as an electroporation generator or ultrasound generator as described above. Further, it may be possible to inject a treatment enhancing agent 54 through the microneedles.

The microneedles 62 may be bipolar between rows, or operate in a monopolar fashion with a ground pad (not shown) located somewhere on the patient. Power may be applied in a rastered fashion where various pairs, or sets of pairs may be activated at certain intervals. The spacing between rows of microneedles would be set for maximum field uniformity, for example in the range of 0.5-5.0 mm apart.

In yet another embodiment, the base 266 of the handpiece 268 may include a suction member 264 for sucking the patient's skin 102 up towards the base using subatmospheric pressures. The suction member includes at least one suction tube 270 that may connect to a mechanical pump (not shown), hand pump (not shown) or other source of subatmospheric pressure.

In one embodiment, the skin 102 is sucked up towards needles 62 that are deployed out of the handpiece 268 before the suction is applied to the skin. Thereafter, suction is applied to the skin and the skin is sucked up towards the base 266 of the handpiece, wherein the needles penetrate through at least the epidermis 102 of the patient to be treated. In another embodiment, the handpiece is placed on the patient in a first configuration, wherein the distal ends of the needles are inside the handpiece. Suction is then applied to pull the skin up against the base of the handpiece. Thereafter, the needles may be deployed into a second configuration where the distal ends of the needles are outside the handpiece, whereby the needles penetrate through at least the epidermis of the patient to be treated. In one embodiment the distal end of the needle may be deployed automatically out of the injection member. Movement of the needles between the first configuration and the second configuration may be controlled by a controller. In at least one embodiment, a motor may be included in the handpiece for automatic deployment of the needles between the first configuration and the second configuration.

FIG. 8A-B show a series of microneedles 62 capable of infusion of treatment enhancing agents 54 and further adapted to extend through the dermal layers 104 (stratum corneum) and into the subdermal layers 106, 108, 110 (FIG. 1) where treatment is desired. For example, application of energy via microneedles 62 according to the present invention can disrupt the septae 108 of the subcutaneous layer, causing an energy assisted subcision and subsequent skin smoothing. In addition, depending on their diameter and the depth of penetration, the microneedles may also deliver enough energy to disrupt the subcutaneous fat 106 cells, sufficient to cause permeabilization of the cell membrane such that treatment enhancing agents can enter the cell and disrupt its function, or sufficient to cause irreversible electroporation leading to cell death. FIG. 8B further depicts an array of microneedles 62 on a conformable pad 60 or reservoir 64 capable of infusion of a treatment enhancing agent 54. In one embodiment, the needles may be arranged in a bipolar configuration. In another embodiment, the needles may be arranged in a monopolar configuration and a grounding pad applied to the patient away from the tissue to be treated.

It is within the scope of the present invention to configure the toroid electrodes 51, 52 and clamp electrodes 41, 42 (FIGS. 2-6) with microneedles 62 that are driven through the dermal layers 104 of the patients' skin by pressure applied by the user or the negative pressure of any suction assistance that is used, for example, negative pressure applied to the annular region 36 of the device 30 (FIG. 2) or to the suction member 264. Further, it may be advantageous to allow the user to place the microneedles at distances and in locations they desire to treat. In doing so, it is within the scope of the invention to provide a template 90 (FIG. 15) through which separate needles 62 could be placed by the user, and depending on the placement chosen, certain energy algorithms provided.

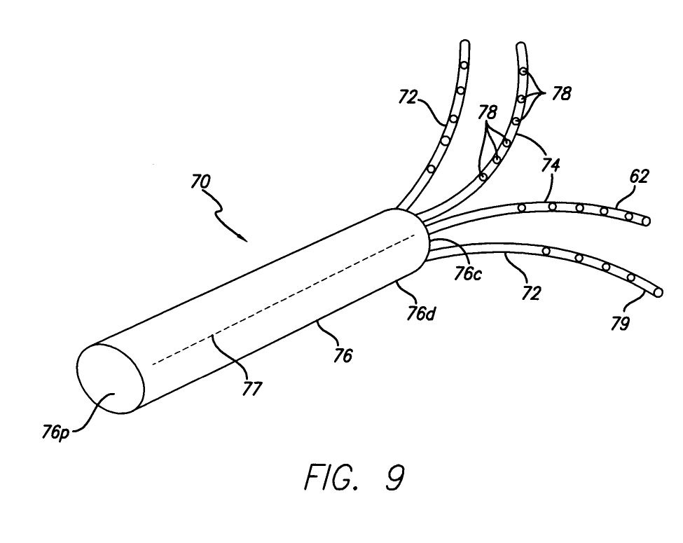

Referring now to FIG. 9, in a further embodiment of the present invention, a catheter device 70 adapted to deploy needles 62, an electrode 72, or electrode array 74 may be provided for insertion through the skin 102, 104 to a targeted subcutaneous structure 106, 108, 110. For example, a fanned electrode array 74 with multiple extending elements or tines 62, 72 may be provided.

The tines 62, 72 may be deployed through the skin 102, 104 through the main catheter shaft 76, and “fan out” in an orientation substantially horizontal (parallel) to the skin surface 102. In embodiments where the tines are also electrodes 72, upon deployment of the tines such that they are substantially parallel to the skin surface and application of energy, the subcutaneous structures such as subcutaneous fat 106 or the fibrous septae 108 may be disrupted. Using multiple tines, it is possible to treat a greater area in a shorter amount of time than is contemplated by devices today. The tines of the electrode device 70 may further be adapted to be hollow to allow injection of treatment enhancing agents 54. The hollow tines may have outlet ports 78 at the distal end 79 as well as along the length thereof.

In one embodiment, the fanned tine array 74 may include a tubular element 70 having a first proximal end 76p, a second distal end 76d adapted for insertion into subcutaneous tissue, and a channel 76c longitudinally disposed therebetween. A plurality of extendable elongated elements 72, 74 having first proximal ends (not shown) and second distal ends 79 disposed within the channel and capable of movement from a first retracted configuration within the channel to a second extended configuration outside of the channel, wherein the distal ends of the elongated elements are farther apart from each other in the extended configuration than in the retracted configuration. In one embodiment, harmonic scalpels may be used in the array. In yet another embodiment, mechanical scalpels or cutting elements may be used in the array.

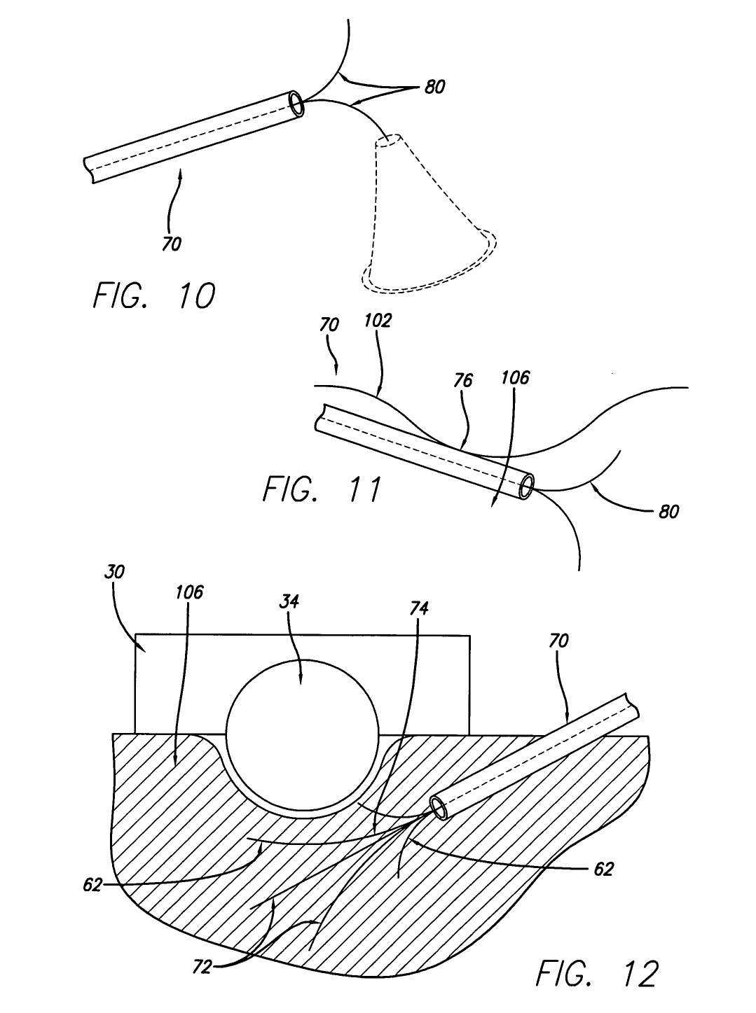

Referring also now to FIG. 10, in yet another embodiment of the present invention the tines are merely sharp cutting elements 80 that do not deliver energy, but when the tines are positioned parallel to the skin surface 102 and are rotated about the longitudinal axis 77 (FIG. 9) of the catheter shaft 76 or retracted in a substantially parallel orientation to the skin, the device 70 can efficiently disrupt multiple septae 108 in one rotation or retraction. In still another embodiment at least one catheter shaft 76 may be adapted for infusion of treatment enhancing agents 54 into the tissue to be treated. In still another embodiment, the needle tip geometry may be configured to shape the energy field for particular tissue disruption effects.

Referring now to FIG. 11, in yet a further embodiment, an active element 80, for example a cutting element, may be deployed at an acute angle to the center axis 77 of the catheter shaft 76. The active region 80 of the device can be collapsed for insertion through the catheter shaft, and then expanded once placed in the subcutaneous space 106, 108, 110. Upon expansion to its cutting configuration, as shown in FIG. 11, the catheter shaft is then oriented parallel to the skin surface 102 and the device 70 is pulled back, catching and cutting the septae 108 in its path. The active region 80 of the device 70 may be, for example, a blunt dissector, a mechanical cutter, or an energy assisted device. Any of the applicable energy modalities may be employed, including radiofrequency energy or resistive heat energy.

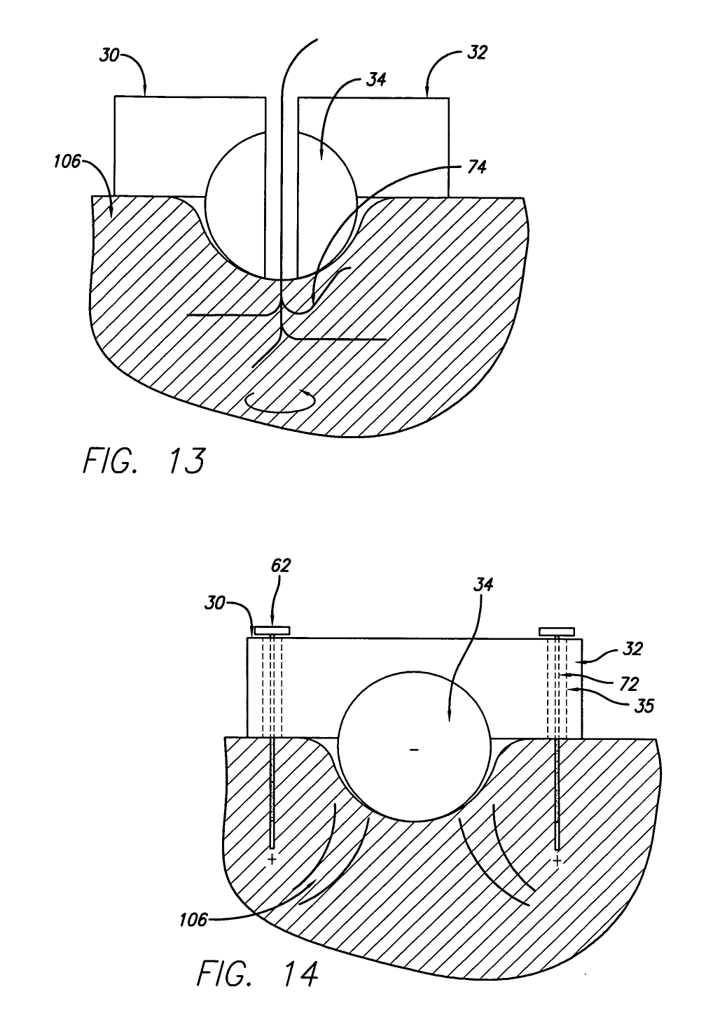

Referring now to FIGS. 12-14, in at least one embodiment, subcutaneous needles 62 or electrodes 72 may be employed with a tissue disruption device 30. In one embodiment, a fan-type electrode 74 is deployed in conjunction with a tissue disruption device 30, for example, the housing 32 and rolling central treatment element 34, clamp 40 or toroidal electrode 50 such as those devices described above and shown in FIGS. 2-6. In yet another embodiment, the fan-type electrode 74 may be deployed independently of the device 30, for example, the rolling central treatment element 34.

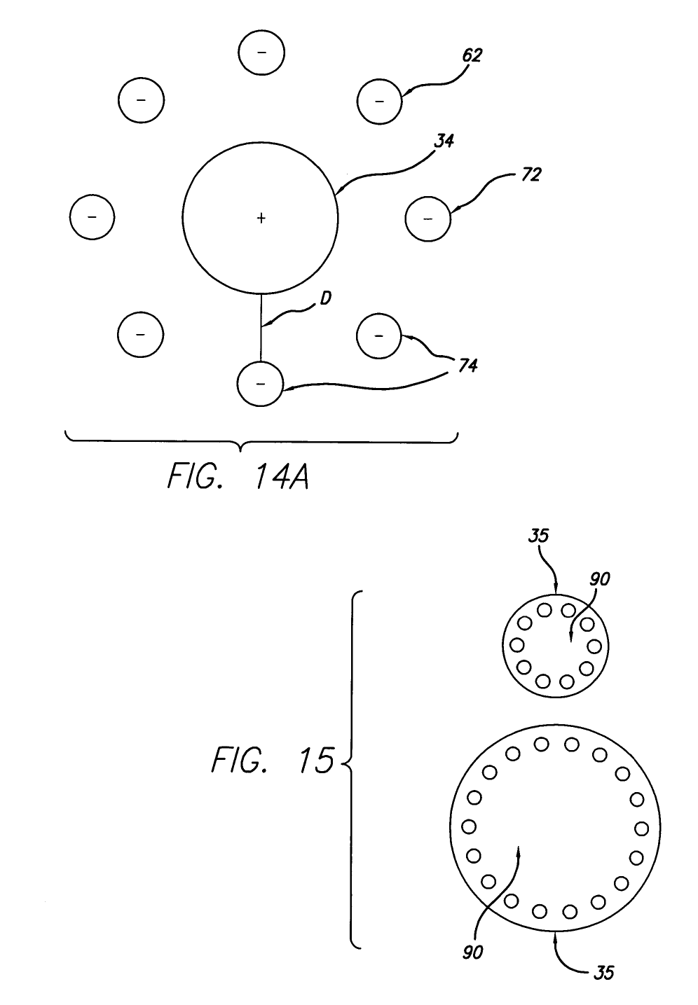

In one embodiment, the fan-type needle electrodes 74 may be oriented such that the electric field they produce is advantageously positioned to target connective tissue such as the fibrous septae 108. Referring to FIGS. 12-13, in at least one embodiment the central treatment element 34 is positively charged and the needle electrodes 62,72,74 are negatively charged. One embodiment shown in FIG. 13 may include deployment of a fan-type electrode 74 through the center of a toroid shaped central treatment element 34. The fan-type electrode may be rotated to mechanically assist the energy disruption of the tissue to be treated. Another embodiment shown in FIG. 14 may include straight needle electrodes 62, 72 deployed in the housing 32 and configured around the edge of the toroid shaped central treatment element 34. The needles may be subcutaneously shaped or configured such that the electrical field lines are oriented vertically or parallel to the septae, wherein the septae may be electrically disrupted. Referring to FIG. 14, the needle electrodes 62, 72 may be electrically insulated proximally with exposed electrically active distal tips. In at least one embodiment the central treatment element 34 is negatively charged and the needle electrodes 62,72,74 are positively charged. Referring now to FIG. 14A, the distance “D” between the positively charged central treatment element 34 and the surrounding negatively charged electrodes 62, 72, 74 may be varied to shape the distribution of the disruptive energy to the tissue to be treated.

Referring also now to FIG. 15, in at least one embodiment, the housing 32 may be configured as a template 90 with channels 35 that guide the insertion of the straight needle electrodes 62, 72. Various size templates may be provided, thereby allowing a variety of insertion patterns for the needle electrodes. Templates 90 can be sized to focus on a particular region, such as over a scarred region, or in cases of severe cellulite, a particular dimple or cluster of dimples. A large cellulite dimple may be treated with a larger template, and a smaller cellulite dimple may be treated with a smaller template. The energy may be adjusted for the particular template that is used to treat a patient.

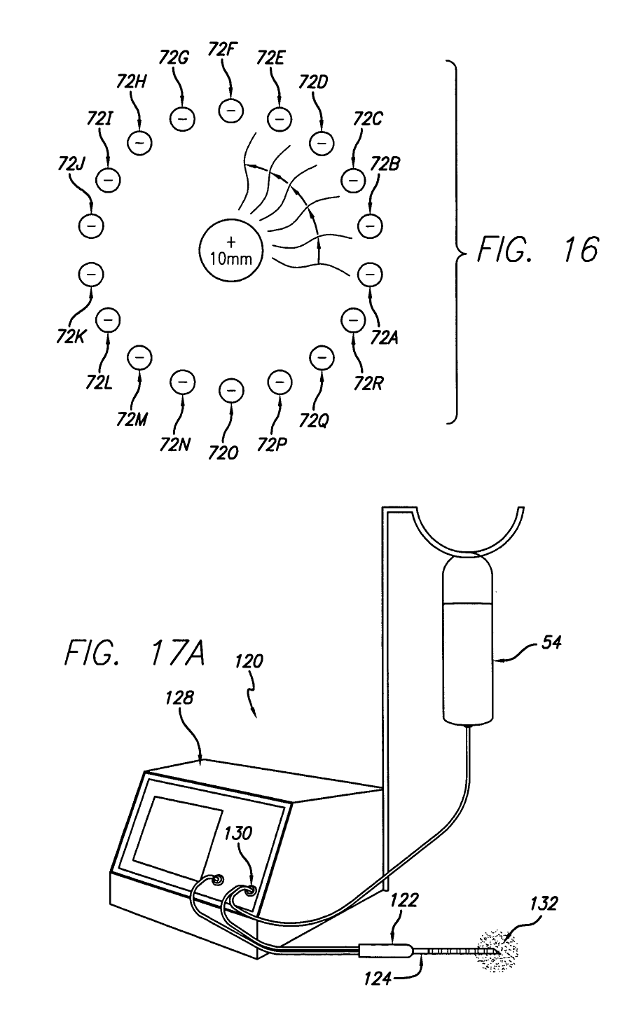

In one embodiment, the central treatment element 34 can act in conjunction with at least one needle electrode 62, 72 to maximize the effective treatment region. Further, the needle electrodes may be placed around the periphery of the housing 32 of the device 30, and energized together, or multiplexed. Referring also now to FIG. 16, in at least one embodiment, the central treatment element may be configured as a positively charged electrode and a plurality of peripherally distributed needle electrodes 72 may be configured as negatively charged electrodes. These polarities are by example only, and it should be understood that the electrode polarities may be switched or modified depending on the type of energy delivered and the desired effect. A template 90 may be used to guide placement of electrodes 72 or needles 62 by the user. As referenced above, depending on the desired volume of tissue to be treated a variety of differently sized and shaped templates may be provided.

Referring more specifically now to FIG. 16, in one embodiment, the energy can be delivered continuously from the central electrode 34, but each peripheral ground electrode 72A-72R is activated in a timed sequence. The peripheral electrodes 72A-72R may therefore be stimulated sequentially. This may reduce muscle stimulation by providing a constant delivery of energy, while also reducing total energy delivery due to the higher impedance of a single ground electrode. Each tissue sector would be energized only a fraction of the time, thereby minimizing tissue heating and thermal damage. If this electrode array was moved slowly over the total area to be treated, an even lower energy therapy may result. In one embodiment, the distance between the central positive electrode and the surrounding peripheral electrodes is 10.0 millimeters. In at least one embodiment, the energy is delivered at 10 ohms, 200 volts, 0.5 amps, and/or 100 watts. In still another embodiment, a 1/20 duty cycle is used in any one area. In yet another embodiment, a higher impedance and lower power is used

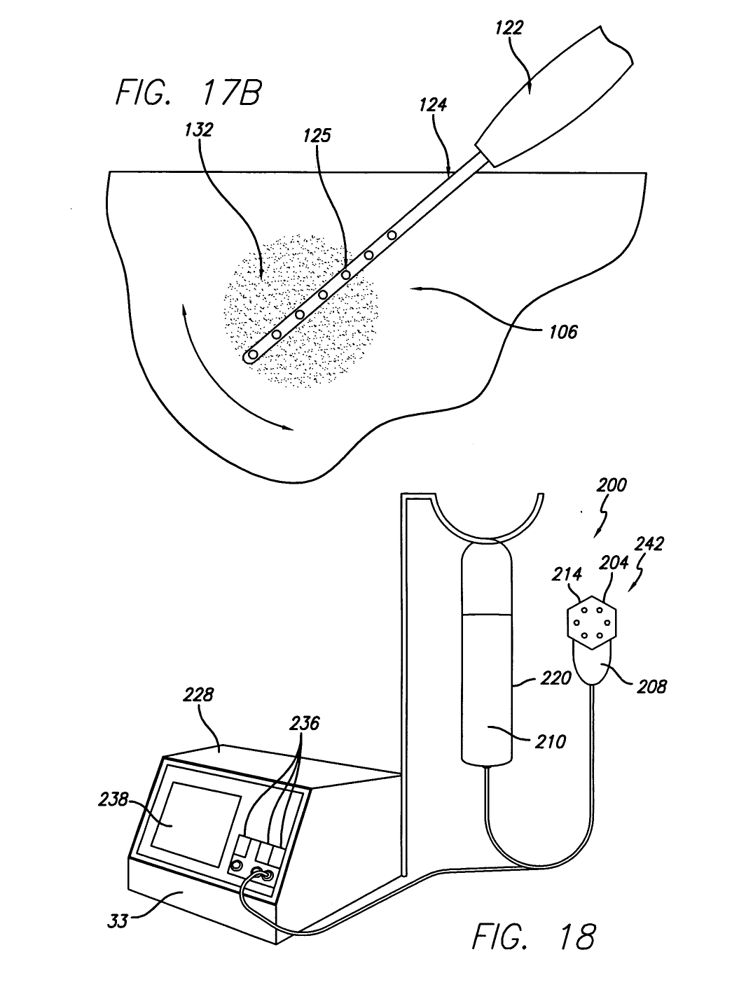

Referring now to FIGS. 17A-17B, in yet another embodiment of the present invention an ultrasound device 120, for example an ultrasound catheter having a handpiece 122 and a treatment shaft 124, is employed to disrupt subcutaneous structures with the application of ultrasound energy. The ultrasound device may include, for example, a harmonic scalpel, or mounting an ultrasound transducer at the tip of a needle cannula. Such a device 120 can be used in conjunction with the infusion of a treatment enhancing agent 54, either through apertures 125 in the treatment shaft itself, or from a separate injection device 56 (FIG. 6) directed to the treatment region, for example, the subcutaneous fat 106. An optional controller 128 may be employed to ensure that the treatment enhancing solution is injected prior to application of energy. Further, similar injection and foaming devices 56, 130 as described above, can be employed to inject microbubbles 132 (agitated saline and the like) to the treatment area. In one embodiment, a harmonic needle or ultrasonic treatment shaft is configured to be swept back and forth under the subcutaneous tissue or cellulite dimple to be treated.

In at least one embodiment, the present invention includes an apparatus for disrupting subcutaneous structures 106, 108 (FIG. 1) in a mammalian patient. The apparatus may include an applicator 30, 70 (FIG. 2, FIG. 9) having one or more energy transmission members 34, 40, 50 or electrodes 72, 74 disposed on a surface of the applicator. In one embodiment, the applicator is configured as a catheter 70 (FIG. 9). The electrodes are adapted to transmit an electrical pulse. The apparatus further includes a pulse generator 33 (FIG. 6) operatively connected to the applicator and adapted to supply an electric pulse of between about 10V and 3000V. The applicator and generator may be configured to disrupt a collagenous subcutaneous structure, for example fibrous septae 108.

In another embodiment (FIGS. 9-13), the applicator is a catheter device 70 adapted to be inserted through the skin 102, 104 of the mammalian patient to a region adjacent the subcutaneous structures 106, 108 to be treated. The catheter or applicator may be positioned at an angle to the targeted collagenous structure 108 to be treated.

In at least one embodiment (FIG. 4), the applicator 50 is a toroidal shape having at least one radius of curvature, and at least one surface.

Referring again to FIG. 2, In yet another embodiment, the applicator 34 is mounted in a housing 32 and is further adapted to move relative to the housing. In yet a further embodiment, the housing is an active electrode and the applicator is a return electrode or ground. In another embodiment, the housing is a return electrode or ground and the applicator is an active electrode. In one embodiment, the applicator is rotatably connected to the housing to allow the applicator to rotate in multiple directions.

Referring again to FIGS. 7A-8C, in another embodiment, at least one surface of an applicator may further include microneedles 62 capable of penetrating the skin of the mammalian patient. The microneedles may include energy transmission elements. In yet another embodiment, the applicator is configured as a conformable pad 60. The conformable pad may further include microneedles extending therefrom, capable of penetrating the skin 102, 104 of a mammalian patient. The applicator may be configured such that one or more electrodes include microneedles capable of penetrating through the skin of the mammalian patient.

In a further embodiment, the invention also includes a method for selective disruption of subcutaneous structures contributing to a skin irregularity in a mammalian body. The method includes providing a energy transmission device having a first 41 and second electrode 42. A pulse generator 33 adapted to produce an electric field between the first and second electrodes is provided. The energy transmission device is positioned at a region adjacent the subcutaneous tissue 106, 108 to be treated and the subcutaneous structure is energized at the cellular level to effect permeabolization of at least one cell so as to disrupt the subcutaneous structure. In one embodiment, the cellular permeabolization is reversible. In another embodiment, the cellular permeabolization is irreversible. In a further embodiment, the irreversible cellular permeabolization is achieved via creation of apoptosis of the intracellular matrix.

Referring again to FIG. 6, in yet another embodiment, the invention includes a method of treating subcutaneous tissue including providing a treatment enhancing agent 54, and delivering the treatment enhancing agent, for example, through an injector 56, in conjunction with the activation of the electric field between a first electrode 34 and a second electrode 32. The treatment enhancing agent may include anesthetics such as lidocaine, vasoconstrictive agents such as epinephrine, hypotonic solutions, hypotonic saline, potassium, agitated saline, microbubbles, and/or microspheres, lidocaine, or a tumescent solution.

In still a further embodiment, a method for treating cellulite includes local delivery of energy to cells of the fibrous septae 108 of the subcutaneous region of a patient. The energy is delivered to the cells under conditions selected to permeabilize the cell membrane of the fibrous septae sufficient to disrupt the septae.

Referring again to FIG. 12, in at least one embodiment, an apparatus for disrupting subcutaneous structures in a mammalian patient includes an applicator 30 having one or more energy transmission members 34 disposed on a surface thereof wherein the energy transmission member is adapted to transmit an energy field. A treatment enhancing agent 54 may be applied to the tissue to be treated 100 in conjunction with the transmission of the energy field. The energy transmission member and the treatment enhancing agent operate to disrupt a collagenous subcutaneous structure 108. The subcutaneous structure may be oriented substantially at an angle to the applicator.

The methods and apparatus discussed herein are advantageous for the disruption and/or destruction of subcutaneous structures 106, 108 in a mammalian body, for the treatment of skin irregularities, and for the treatment of other disorders such as excess adipose tissue, cellulite, and scarring. The devices and methods may include energy mediated applicators, microneedles, catheters and subcutaneous treatment devices for applying a treatment non-invasively through the skin, less invasively through the skin, or minimally invasively via a subcutaneous approach. Various agents known in the art and discussed herein may assist or enhance these procedures for treatment of subcutaneous tissues.

In one embodiment, the present invention includes an apparatus for treating soft tissue. In another embodiment, the present invention includes a method for treating fibrous tissue. In one embodiment, the present invention further includes a method and apparatus for treating a subcutaneous fat layer 106 including fat cells and septae 108. In one embodiment, the present invention further includes a method and apparatus for treating cellulite. The present invention may be useful for a temporary reduction in the appearance of cellulite or the permanent reduction of cellulite. The invention may also be used as an adjunct to liposuction. The invention further provides for a subcutaneous infusion and dispersion of fluid to temporarily improve the appearance of cellulite. The invention may also be advantageous for a removal of benign neoplasms, for example, lipomas.

In at least one embodiment, the present invention is directed to methods and apparatus for targeting and disrupting subcutaneous structures, such as collagen, connective tissue, adipose tissue (fat cells) and the like (collectively “target tissue” or “subcutaneous structures”) in order to improve the aesthetic appearance of the targeted region. Targeted regions may consist of any surface or contour of the human form that it is desirable to enhance, including the face, chin, neck, chest, breasts, arms, torso, abdominal region (including pelvic region), thighs, buttocks, knees and legs. The target tissue may include the connective tissue or septae of the region, or the underlying tissues that may exacerbate the unwanted body contour, such as subdermal and deeper fat deposits or layers. Skin irregularities refer to conditions that decrease a person's satisfaction with their outward appearance, such as cellulite, scarring, or fat deposits or excess fat in certain regions, such as neck, chin, breasts, hips, buttocks, abdomen, arms and the like.

The term enhancing agent 54 as used herein refers to at least one of an exogenous gas, liquid, mixture, solution, chemical, or material that enhances the disruptive bioeffects of an energy delivery system 33 when applied on tissue. One example of an enhancing agent is an enhancing solution. In one embodiment, the enhancing solution contains exogenous gaseous bodies, for example, microbubbles 132. The enhancing agent or solution may include, for example, saline, normal saline, hypotonic saline, a hypotonic solution, a hypertonic solution, lidocaine, epinephrine, a tumescent solution, and/or microbubble solution. Other enhancing agents are described in more detail herein. In one embodiment, the present invention is an assembly that further includes an agitation system 56 configured to agitate and/or mix an enhancing agent solution and an injection member 56, 122 configured to inject the solution. In at least one embodiment, the assembly may also include a container for storing the solution, for example a reservoir 64 for storing the solution therein. The reservoir may be an IV bag known in the art.

Referring now to FIG. 18, in one embodiment an assembly 200 includes a energy delivery system 33. The physician may prepare and hang an enhancing solution 210, and the assembly mixes, injects and applies energy to the tissue to be treated according to a pre-programmed or a user defined algorithm. The algorithm may be programmed into a controller 228. The controller may be included in a unitary assembly with the other components, or may be a separate unit configured to communicate with the other components of the assembly. In at least one embodiment, the controller includes a processor and memory. In at least one embodiment, the controller may also include inputs 236, for example, electrical switches, buttons, or keypad. In at least one embodiment, the controller may also include outputs 238, for example, LED lights, an LCD screen, gauges, or other screens and output indicators known in the art. In other embodiments, the inputs 236 and outputs 238 may be separate from the controller but in electrical communication with the controller. The assembly is configured to transport the enhancing solution 210 from a reservoir 220 to an agitator 208, where the solution is mixed and agitated. The agitator 208 that may be included in a unitary handpiece 242. The assembly is configured to thereafter inject the solution into the patient using an injection member 214. The assembly is also configured to apply energy to the injected tissue 100 to be treated using the energy delivery device 204 included in the handpiece. The handpiece may be configured as a housing 32 with a central treatment element 34, for example, the tissue disruption device 30 illustrated in FIG. 2. In one embodiment, at least one hypodermic needle 62 is disposed in the solution injection member 214. In yet another embodiment, the solution injection member may be configured with retractable needles 62.

The present invention also includes a variety of treatment enhancing agents 54 that are biocompatible with subcutaneous injection into the subcutaneous fat 106 of a patient. In one embodiment, the solution is a tumescent solution. Tumescent solutions are specially adapted to provide for the application of local anesthesia and are well known in the art. Tumescent solutions may include a variety of medicated solutions. One example of a tumescent solution is a solution that includes 1000 milliliters of normal saline with 2% lidocaine, 30 ml. (600 mg) of epinephrine, and one mole (12.5 ml or 12.5 mg.) of sodium bicarbonate. At least one other example of a tumescent solution is a solution that includes 1000 milliliters of normal saline, 50 ml of 1% lidocaine, and 1 cc. of 1:1000 epinephrine. These additives are commercially available. Tumescent solutions may decrease bleeding at the treatment site and provide for local anesthetic effects that decrease pain during and after the procedure.

In one embodiment, the enhancing solution 54 is a normal saline solution. In yet one further embodiment, the enhancing solution is a hypotonic solution. In yet one other embodiment, the solution is a solution including microbubbles or nanobubbles. The solution may be agitated between two syringes one or more times to produce a solution including microbubbles. Several solutions including microbubbles or nanobubbles are commercially available, as described in detail elsewhere herein.

The enhancing agent included depends on the desired effects, some of which are detailed below. For example, enhancing agents may be transmitted transdermally, or via injection into the tissue to be treated. Treatment enhancing agents include, anesthetics such as lidocaine, a surfactant, vasoconstrictive agents such as epinephrine, hypotonic saline, potassium, agitated saline, microbubbles, commercially available ultrasound contrast agents, microspheres, adipocytes, fat, autologous tissues (e.g. lysed fat cells to produce clean adipocytes to form a tissue graft to minimize hostile response from the body), PLLA, hydroxyappetite. Treatment enhancing agents may be delivered prior to, during or following the application of acoustic waves to the subcutaneous tissue.

In one embodiment, power to the solution injection member 214 is included within the solution injection member. In another embodiment, power to the solution injection member is located externally to the solution injection member. For example, power to the solution injection member may be supplied by the controller 228. In at least one embodiment, algorithms controlling the injection volume, depth, timing, and synchronization of injection with the application of ultrasound may be included in memory and/or a processor included within the solution injection member. In at least another embodiment, algorithms controlling the injection volume, depth, timing, and synchronization of injection with the application of ultrasound may be included in memory and/or a processor located externally to the solution injection member, for example, in the controller.

In one embodiment, the solution injection member 214 includes at least one hypodermic needle 62. The hypodermic needle has a proximal end connected to the solution injection member and a distal end configured for penetrating into the targeted region 100 to be treated. The distal ends of the needles may be beveled (not shown) as known in the art for less traumatic penetration into the skin. In one embodiment, the needles may include microneedles. In at least one embodiment, the needles may be pyramid shaped (not shown). In one further embodiment, the solution injection member includes a plurality of hypodermic needles. The hypodermic needle has a tubular channel having a central lumen configured for flow of the solution through the needle and into the tissue. In one embodiment, the solution injection member includes an actuation element (not shown) for moving the hypodermic needle from a position inside the solution injection member to a position wherein the needle may penetrate through the epidermis 102 and into the subcutaneous tissue to be treated. In one embodiment the needles are configured to penetrate at least into the subcutaneous fat 106. In yet one other embodiment, the needles are configured to penetrate into the deep fat layer 110.

The injection needles diameter may range in size from 40 gauge to 7 gauge. In one embodiment the injection needles include size 30 gauge. In another embodiment the injection needles include size 28 gauge. In one further embodiment the injection needles include size 25 gauge. In one additional embodiment the injection needles include size 22 gauge. In yet another embodiment the injection needles include size 20 gauge. In still one other embodiment the injection needles include size 18 gauge. The needles may all be of one length or may be of different lengths. In one embodiment, the length of the needles are between 2.0 mm long and 10.0 cm long. In one embodiment, the length of the needles are less than 5 mm long. In another embodiment, the length of the needles are in the range of 5.0 mm to 2.0 cm. In one other embodiment, the length of the needles are in the range of 1.0 cm to 3 cm. In yet another embodiment, the length of the needles are in the range of 2.0 cm to 5 cm. In still another embodiment, the length of the needles are in the range of 3.0 cm to 10.0 cm.

In at least one embodiment, the injection needles 62 include microneedles. In one embodiment, the diameter of the microneedles may be in the range of 20 microns to 500 microns. In one embodiment, the length of the microneedles may be in the range of 100 microns to 2000 microns. In at least one embodiment, the needles are long enough to reach from the epidermis 102 to the deep fat layer 110. In at least one further embodiment, the needles are long enough to reach from the epidermis to the muscle layer 26. In at least one embodiment, to increase patient comfort, further anesthesia may be applied to the area to be treated using topical anesthetic creams or gels, local hypothermia, or regional blocks. Topical anesthetic may be the only anesthetic necessary and may take the place of any lidocaine used as an enhancing agent.

In one embodiment, the needles 62 may be long enough to extend into the subcutaneous tissue 106 a distance of 0.2 mm to 40 mm from the skin surface, depending on the target tissue to be treated. The needle is long enough to allow the distal end 226 of the needle to extend at least through stratum corneum. For example, to treat cellulite a depth of penetration from 1.0 mm-5.0 mm may be desired, and for deeper subcutaneous fat, a depth of 3.0 mm-40 mm. One or more hypodermic needle may be moved to various depths manually or automatically by the controller 228. In at least one embodiment, the needles are long enough to reach from the epidermis 102 to the deep fat layer 110. In at least one further embodiment, the needles are long enough to reach from the epidermis 102 to the muscle layer 26.

In at least one embodiment, the present apparatus is configured to provide staged depths of injection from the deeper tissue layer to the more superficial tissue layer with application of energy to the tissues between each stage of injection. One or more hypodermic needle may be moved to various depths manually or automatically by the controller 228 wherein the tissue can be treated at staged depths as described further below.

The assembly 200 is configured to allow activation of the energy delivery system 33 at various times after injection of the solution by the injection member 214. In at least one embodiment, the controller 228 may be used to synchronize the timing of the energy application to the tissues following the injection of the solution into the tissue to be treated. In one embodiment, the injection member is further configured with an on switch to start at least the injection of the solution into the tissue to be treated. In at least one embodiment, the injection member may be configured with a stop switch to stop the injection and/or withdraw the needles 62 from the patient.

In yet another embodiment of the invention, the assembly 200 includes a cooling module (not shown). Injection into the skin of a patient may commonly be associated with the side effect of discomfort, swelling, bleeding, scarring or other undesired effects. Furthermore, the disruption of subcutaneous tissues treated by the present invention may also result in some side effects common to many cosmetic or dermatologic treatment. The use of a cooling module reduces the side effects of the treatment with the invention. Cooling of the tissues reduces bleeding, swelling, and discomfort. The cooling module may include any of the many known methods of cooling tissue known in the art. In at least one embodiment a portion of the cooling module may be included with the handpiece 242. One advantage of the cooling module is to assist in treatment or prophylaxis of discomfort, swelling, scarring and other undesired effects associated with treatments of the present invention. In at least one other embodiment, the cooling module may be included in the assembly as a separate module. In yet another embodiment, the enhancing solution may be cooled prior to injection into the tissue to be treated.

The handpiece 242 may be provided in different sizes that are configured to treat different subcutaneous abnormalities or different severities of subcutaneous abnormalities. One handpiece may have a more dense pattern of needles 62 than another. For example, a more severe area of cellulite may be treated with the handpiece having the more dense pattern of needles. In at least one embodiment having a disposable handpiece, a security chip (not shown) may be provided in the handpiece to prevent re-use of the handpiece on other patients, thereby preventing the spread of disease, for example, hepatitis or aids. The security chip may also be included to prevent counterfeit handpieces from being distributed and used on patients.

Yet another factor in producing consistent results may be a volume of injected solution per skin surface area of a location to be treated. In one embodiment the volume of injection is in the range of about 0.1 cc/sq cm of skin surface area in the location to be treated to about 2.0 cc/sq cm of skin surface area in the location to be treated. In another embodiment the volume of injection is in the range of about 0.25 cc/sq cm of skin surface area in the location to be treated to about 1.5 cc/sq cm of skin surface area in the location to be treated. In yet one other embodiment the volume of injection is in the range of about 0.5 cc/sq cm of skin surface area in the location to be treated to about 1.0 cc/sq cm of skin surface area in the location to be treated. However, the above volumes to be injected are exemplary only, and may be varied depending on the pain tolerance of the individual patient treated and the depth of the fat layer in the location to be treated.

Still another factor in producing consistent results may be the rate of injection of the solution into the tissue to be treated. In one embodiment, the rate of injection of the solution is in the range of about 0.01 cc/second to about 1.0 cc/second. In another embodiment, the rate of injection of the solution is in the range of about 0.02 cc/second to about 0.5 cc/second. In still another embodiment, the rate of injection of the solution is in the range of about 0.05 cc/second to about 0.2 cc/second. However, the above rates of injection are exemplary only, and may be varied depending on the pain tolerance of the individual patient treated and the pathology of the fat layer in the location to be treated.

The invention includes a method of disrupting subcutaneous tissue. The method may includes disposing at least one enhancing agent 54 to the subcutaneous tissue 100 to be treated. The enhancing agent may be included in a solution. The solution may be injected into the subcutaneous fat 106 through at least one hypodermic needle 62. The needle may then be withdrawn leaving the enhancing agent disposed in the subcutaneous tissue for a period of time. An energy delivery system 33 may then supply energy to the tissue to be treated, wherein the subcutaneous fat 106 and/or the fibrous septae 108 in proximity to the enhancing agent are disrupted.

One factor in the amount of energy transmitted to the tissue and the bioeffects on the tissue may be the length of time that the injected solution is in the tissue before the disruptive energy is applied to the tissue. In one embodiment, the injected solution is infiltrated into the tissue about 10 minutes to about 30 minutes before the application of the disruptive energy. In yet another embodiment, the injected solution is infiltrated into the tissue about 1 minute to about 10 minutes before the application of the disruptive energy. In still another embodiment, the injected solution is infiltrated into the tissue about 1 second to about 1 minute before the application of the disruptive energy. In at least one further embodiment, the injected solution is infiltrated into the tissue about 50 milliseconds to about 1000 milliseconds before the application of the disruptive energy. In at least one other embodiment, the disruptive energy is applied to the tissue to be treated about simultaneously with the injection of the solution.

The duration of disruptive energy exposure may also determine the bioeffects of the disruptive energy on the tissue. In one embodiment, disruptive energy is applied to the tissue to be treated 100 for a duration of about 10 seconds. In another embodiment, disruptive energy is applied for a duration of about 30 seconds. In yet another embodiment, disruptive energy is applied for a duration of about 1 minute. In yet a further embodiment, disruptive energy is applied for a duration of about 2 minutes. In at least one other embodiment, disruptive energy is applied for a duration of about 5 minutes. In yet one other embodiment, disruptive energy is applied for a duration of between about 5 minutes and 20 minutes. In still one other embodiment, disruptive energy is applied for a duration of between about 20 minutes and one hour.

Tumescent solutions are specially adapted solutions that provide for the application of local anesthesia, for example, during liposuction procedures. Tumescent solutions are well known in the art. Tumescent solutions employ a variety of medicated solutions. In one embodiment, the tumescent solution includes 1000 milliliters of normal saline with 2% lidocaine, 30 ml. (600 mg) of epinephrine, and one mole (12.5 ml or 12.5 mg) of sodium bicarbonate. In at least one other embodiment, the tumescent solution is a solution that includes 1000 milliliters of normal saline, 50 ml of 1% lidocaine, and 1 cc of 1:1000 epinephrine. These additives are commercially available. In one embodiment, the tumescent solution may be mixed in the agitator 208. In another embodiment, a premixed or commercially available tumescent solution may be used. Tumescent solutions may decrease bleeding at the treatment site and may provide for local anesthetic effects that decrease pain during and after the procedure. In at least one embodiment, enhancing agents may also be included in the tumescent solution. In at least one embodiment, the enhancing solution 54 to be injected is a hypotonic solution.

In at least one further embodiment, treatment at various subcutaneous tissue depths is performed in stages. Each injection may be followed by an application of disruptive energy to the tissue to be treated. For example, in a first stage, a deep injection of solution is performed followed by an application of disruptive energy to the deeper layer. In a second stage, a more superficial injection of solution is performed followed by an application of disruptive energy at the more superficial layer. Multiple stages of injection of solution at gradually more superficial depths may be performed with the application of disruptive energy, for example, disruptive energy after each injection of solution. In one embodiment, each subsequent stage of injection is performed at a depth about 0.5 ruin to 2.0 cm more superficial than the previous stage of injection. In one embodiment, each subsequent stage of injection is performed at a depth about 0.5 mm more superficial than the previous stage of injection. In another embodiment, each subsequent stage of injection is performed at a depth about 1.0 mm more superficial than the previous stage of injection. In yet one additional embodiment, each subsequent stage of injection is performed at a depth about 2 mm more superficial than the previous stage of injection. In another embodiment, each subsequent stage of injection is performed at a depth about 5 mm more superficial than the previous stage of injection. In yet another embodiment, each subsequent stage of injection is performed at a depth about 1.0 cm more superficial than the previous stage of injection. In yet one further embodiment, each subsequent stage of injection is performed at a depth about 1.5 cm more superficial than the previous stage of injection. In one further embodiment, each subsequent stage of injection is performed at a depth about 2.0 cm more superficial than the previous stage of injection. In yet one other embodiment, infiltrating the subcutaneous tissue is performed in stages at depths of about 30 mm, about 25 mm, and about 20 mm. In one further embodiment, infiltrating the subcutaneous tissue is performed in stages at depths of about 15 mm, about 10 mm, about 5 mm and about 2 mm. In at least one embodiment, one series of disruptive energy may be applied to the tissue after all depths have been injected, rather than the disruptive energy being applied between injections.

In one embodiment, the tissue to be treated may be injected between the dermal layer 104 and the deep fat layer 110. In another embodiment, the tissue to be treated may be injected between the superficial fat layer 106 and the muscle layer 26. In yet one other embodiment, the tissue to be treated may be injected between the dermal layer 104 and the muscle layer 26. In one embodiment, the tissue to be treated may be injected at depths of about 2 mm to 4.0 cm. In one embodiment, the tissue to be treated may be injected at depths of about 0.5 mm. In at least one embodiment, the tissue to be treated may be injected at depths of about 1.0 mm. In yet one additional embodiment, the tissue to be treated may be injected at depths of about 1.5 mm. In one embodiment, the tissue is injected and treated at a depth of about 2 mm. In another embodiment, the tissue is injected and treated at a depth of about 5 mm. In yet another embodiment, the tissue is injected and treated at a depth of about 1.0 cm. In yet one further embodiment, the tissue is injected and treated at a depth of about 1.5 cm. In one further embodiment, the tissue is injected and treated at a depth of about 2.0 cm. In one further embodiment, the tissue is injected and treated at a depth of about 2.5 cm. In one further embodiment, the tissue is injected and treated at a depth of about 3.0 cm. In one further embodiment, the tissue is injected and treated at a depth of about 3.5 cm. In one further embodiment, the tissue is injected and treated at a depth of about 4.0 cm. In one embodiment, a single depth of injection or tissue infiltration is performed. In at least one other embodiment, more than one depth of injection or infiltration is performed.

The time lapse between the injection of the solution and the application of the disruptive energy may be in the range of about zero seconds to about one hour. An automatic controller 228 may be used to synchronize the timing of the application of disruptive energy following the injection of the solution 54. In one embodiment, the application of the disruptive energy may be about simultaneous with the injection of the solution. In one embodiment, the injection may be performed less than about 5 seconds before the application of the disruptive energy. In another embodiment, the injection is performed about 5 seconds to about 20 seconds before the application of the disruptive energy. In one further embodiment, the injection is performed about 20 seconds to about 60 seconds before the application of the disruptive energy. In yet one other embodiment, the injection is performed about one minute to about five minutes before the application of the disruptive energy. In one further embodiment, the injection is performed about 5 minutes to about 15 minutes before the application of the disruptive energy. In yet one more embodiment, the injection is performed about 15 minutes to about 30 minutes before the application of the disruptive energy. In yet another embodiment, the injection is performed about 30 minutes to about 60 minutes before the application of the disruptive energy.

Yet one further factor in producing consistent results may be the duration of dispersing the solution in the tissue with energy before applying the disruptive energy. In one embodiment, ultrasound may be used to disperse the solution in the tissue to be treated. In one embodiment, the duration of dispersing the solution in the tissue with energy before applying the disruptive energy is about 1 second to 5 seconds. In another embodiment, the duration of dispersing the solution in the tissue with energy before applying the disruptive energy is about 5 seconds to 30 seconds. In one further embodiment, the duration of dispersing the solution in the tissue with energy before applying the disruptive energy is about 30 seconds to 60 seconds. In still another embodiment, the duration of dispersing the solution in the tissue with energy before applying the disruptive energy is about 1 minute to 5 minutes.

In one embodiment, following disruption of the treated tissue, the disrupted tissue may be left in the patient, for example, to be absorbed by the patient's body. In another embodiment, the disrupted tissue may be removed from the patient's body, for example, by liposuction.

In one embodiment, the electrodes may be placed on the skin and are configured to have minimal edge effect in order to avoid any undesired surface burns. In yet another embodiment, subdermal needle electrodes may be configured to concentrate the energy field strength to specific locations adjacent the distal end of at least one of the needles. For example, a pyramidal or beveled distal tip needle would tend to have very high edge effects adjacent the distal end of the needle. In still another embodiment, arranging an array of needles, for example arranging needles side by side, would result in a plurality of high field strength tissue treatment points, thereby causing focal tissue ablation across a larger region of tissue to be treated. As the body heals and remodels the treated tissue, these tissue treatment points may be reabsorbed and the disrupted fat cells removed. This may be similar to the type of remodeling done following treatments including high intensity focused ultrasound arrays wherein focal burns are created in treated tissue with islands of healthy tissue to facilitate healing and transport. In at least one further embodiment, blunt needle electrodes may be included, thereby result in a larger area of treatment disruption effect in the treated tissues.

The invention may be combined with other methods or apparatus for treating tissues. For example, the invention may also include use of skin tightening procedures, for example, Thermage™ available from Thermage Corporation located in Hayward, Calif., Cutera Titan™ available from Cutera, Inc. located in Brisbane, Calif., or Aluma™ available from Lumenis, Inc. located in Santa Clara, Calif.

The invention may be embodied in other forms without departure from the spirit and essential characteristics thereof. The embodiments described therefore are to be considered in all respects as illustrative and not restrictive. Although the present invention has been described in terms of certain preferred embodiments, other embodiments that are apparent to those of ordinary skill in the art are also within the scope of the invention. Accordingly, the scope of the invention is intended to be defined only by reference to the appended claims.

We claim:

1. A medical device for disrupting subcutaneous tissue, comprising:
an electrical field generator;
at least two electrodes electrically connected with the electrical field generator; and
an injection module configured to inject a treatment enhancing solution into the subcutaneous tissue to be treated.

2. The medical device of claim 1, wherein at least one electrode is adapted for insertion into the subcutaneous tissue to be treated and at least one other electrode is adapted for application to the epidermis of a patient to be treated.

3. The medical device of claim 1, wherein at least two electrodes are adapted for application to the epidermis of a patient to be treated.

4. The medical device of claim 1, wherein at least two electrodes are adapted for insertion into the subcutaneous tissue to be treated.

5. The medical device of claim 1, wherein one of the at least two electrodes is configured as a ground electrode.

6. The medical device of claim 1, wherein the at least two electrodes are configured as bipolar electrodes.

7. The medical device of claim 1, wherein one of the at least two electrodes is generally torroidal in shape.

8. The medical device of claim 1, wherein one of the at least two electrodes is generally cylindrically shaped.

9. The medical device of claim 1, wherein the electrical field generator is an electroporation generator.

10. The medical device of claim 1, further including a housing, wherein one of the at least two electrodes is disposed in the housing.

11. The medical device of claim 10, wherein at least one electrode is configured as a central treatment element disposed in the housing, and an annular area is disposed between the central treatment element and the housing.

12. The medical device of claim 11, wherein the annular region is configured for connection with a source of negative pressure, whereby the housing is adapted for contact with the skin overlying the area to be treated.

13. The medical device of claim 11, wherein the central treatment element is recessed into the housing.

14. The medical device of claim 11, wherein the central treatment element is adapted to roll over the skin of a patient to be treated.

15. The medical device of claim 1, further including a pad having microneedles connected to the injection module, wherein the pad is adapted to conform to the skin of a patient to be treated.

16. The medical device of claim 15, wherein the pad further includes a reservoir and an actuation element for deploying the microneedles.

17. The medical device of claim 15, wherein at least one of the microneedles is configured as one of the at least two electrodes.

18. The medical device of claim 1, further including a catheter device adapted to deploy tines to a subcutaneous region to be treated.

19. The medical device of claim 18 wherein the tines are selected from the group consisting of needles, electrodes, and cutting elements.

20. A subcutaneous tissue disruption device, comprising:
a tubular element having a first proximal end, a second distal end adapted for insertion into subcutaneous tissue, and a channel longitudinally disposed therebetween; and
a plurality of extendable elongated elements having first proximal ends and second distal ends disposed within the channel and capable of movement from a first retracted configuration within the channel to a second extended configuration outside of the channel, wherein the distal ends of the elongated elements are farther apart from each other in the extended configuration than in the retracted configuration.

21. The subcutaneous tissue disruption device of claim 20, wherein the plurality of extendable elongated elements are selected from the group consisting of needles, electrodes, and cutting elements.

22. The subcutaneous tissue disruption device of claim 20, wherein the plurality of extendable elongated elements are geometrically configured to shape an energy field for a biological tissue disruption effect.

23. A method for selective disruption of subcutaneous structures, comprising:
providing a first electrode and a second electrode;
disposing the first electrode adjacent to the tissue to be treated;
connecting the first electrode and the second electrode to an energy delivery system, the energy delivery system being configured to produce an electrical current between the first and the second electrode; and
providing electrical current between the first electrode and the second electrode, thereby increasing permeability of at least one cell.

24. The method for selective disruption of subcutaneous structures of claim 23, wherein at least the first electrode is geometrically configured to shape an energy field for a biological tissue disruption effect.

25. The method for selective disruption of subcutaneous structures of claim 23, further including rolling a central treatment element disposed within a housing over the tissue to be treated, wherein the first electrode is disposed in the central treatment element.

26. The method for selective disruption of subcutaneous structures of claim 25, further including providing less than atmospheric pressure to an annular area disposed around the central treatment element.
